(19) United States
(12) Patent Application Publication
(10) Pub. No.: US 2012/0203288 A1
(43) Pub. Date: Aug. 9, 2012

(54) SPINAL FIXATION SYSTEM AND SCREWDRIVER TOOL FOR USE WITH THE SAME

(76) Inventors: Robert Lange, Paris (FR); Andreas Bihl, Zurich (CH)

(21) Appl. No.: 13/500,146

(22) PCT Filed: Oct. 5, 2010
(86) PCT No.: PCT/US10/02675

§ 371 Date: Apr. 4, 2012

Related U.S. Application Data
(60) Provisional application No. 61/272,526, filed on Oct. 5, 2009.

(51) Int. Cl. A61B 17/86; A61B 17/56

(52) U.S. Cl. 606/305; 606/104

(57) ABSTRACT

A spinal fixation system that utilizes a composite rod to which polyaxial pedicle screw/tulip assemblies are secured, a screwdriver that permits independent threading of a guide member to the tulip and independent threading of the pedicle screw, together with interengaging conical surfaces that true the screwdriver with the pedicle screw. In preferred embodiments, the screwdriver includes an elongated drive shaft having a handle end for imparting rotation and a pedicle screw engaging end, a cylindrical guide member rotatably mounted about said drive shaft, a knob at one end of said guide member for rotating same and a threaded tulip engaging end at the other end thereof. A grip is preferably sleeved around the cylindrical drive member and is longitudinally movable between proximal and distal positions wherein its distal end uncovers and covers, respectively, the screw engaging unit.

### CROSS-REFERENCE TO RELATED APPLICATION

This application is related to and claims priority benefits under 35 USC §119(e) from U.S. Provisional Application Ser. No. 61/272,526 filed on Oct. 5, 2009, the entire content of which is expressly incorporated hereinto by reference.

### FIELD

The embodiments disclosed in this application relate generally to tools especially adapted for use with a spinal fixation system that includes a composite rod and a screw/tulip assembly.

### BACKGROUND AND SUMMARY

A surgeon undertaking a spinal fixation installation has four concurrent goals:
(1) Correcting the spinal difficulty, such as degeneration or deformity. To achieve this, the spinal fixation system must apply a corrective force upon the spine.
(2) Stabilizing the spinal segments to be treated so that the correction and alignment is maintained. To achieve this, the spinal fixation system must allow some deflection and then return resiliently due to inherent memory to the form desired by the surgeon as the patient moves and normal body stress are exerted upon it.
(3) Stimulating the spinal bones into which the spinal fixation system is attached as the patient moves. For this, a spinal fixation system must be elastic enough to allow stress to pass through adjacent and connected bone that will encourage bone growth and then return to the original corrective form while at the same time not being so flexible as to over stress the bone and cause micro stress fractures.
(4) To provide improved observation of bone growth during the curative stage and that phase of bone renewal that need to occur through the patient's lifetime.

Elements of the correction, stabilization, stimulation and bone observation framework to attain these goals in combination with the implements to establish and secure that framework are provided by the embodiments disclosed herein.

In the spinal stabilization arts, a common procedure is to first secure a series of polyaxial screws in the appropriate vertebrae bones through a tubular portion of the vertebra bone called the pedicle. These polyaxial screws are then connected to a rod carrying and securing a unit referred to as a tulip. The tulips, as is well-known in the field, are made to pivot in relation to the pedicle screw easing the assembly of the rod to the pedicle screw and tulip. Hence these types of screws are often called polyaxial screws, a term well-known in the art that refers to screws having a pivoting assembly tulip. For screw insertion, the polyaxial pedicle screw is secured to a special insertion screwdriver that allows the pedicle screw and screwdriver to be along the same axis that the screw will be threaded into bone. The special insertion screwdriver is used to thread the pedicle screw into the desired tubular pedicle of the vertebral bone.

A successful construct in part depends on a safe and secure placement of the pedicle screw in the pedicle of the vertebral bone. Proper screw placement provides optimal screw anchorage to allow corrective, stabilizing and stimulating forces to pass through the fixation system and vertebral bone. While this procedure is now commonly performed, a proper, safe and secure pedicle screw placement remains difficult for the surgeon and potentially dangerous for the patient. Beyond pedicle borders are located nerve roots and spinal vascular structures. These can be easily damaged by a threading screw should the screw escape the pedicle boundaries during insertion. As the screw is threaded into the pedicle bone, the surgeon cannot directly see the tubular pedicle. The pedicle's exact boundaries and orientation are mentally imaged by the surgeon who mentally compares bone land marks with previously viewed x-rays. X-rays are only two dimensional and the proper screw placement requires three dimensional situational awareness and control. To this end, as known in the art, the surgeon uses various probes to palpate the pedicle's outer boundaries and verify that any pre-screw probing has not violated safe boundaries of the pedicle. The surgeon also uses the tactile feel of bone density as the screw passes the inner portions of the pedicle bone that are soft and approaches the outer borders that are hard. Due to the different inner and outer densities of bone, the resistance of the bone being threaded with a pedicle screw can change and this can be the surgeon's indication of proper and safe or incorrect and unsafe screw placement.

One of the objectives of the embodiments disclosed herein is to improve the surgeon's security while threading pedicle screws by ensuring a true alignment of the screw to the instruments rotational axis, to prevent instrument loosening in relation to the screw, and to provide a better screw to instrument interface that propagates the vibrations from bone screw to the special screwdriver while the screw is threaded through the bone.

Systems of computerized navigation that render the screw placement in two planes are available to the surgeon so he can determine if the screw has remained in the safe and secure borders of the pedicle. This is done by using preoperative x-rays of the vertebrae to build a virtual vertebra in the computer. These systems use various types of sensors in the operating room, to calculate the screw position in the vertebrae by taking continuous measurements of the instrument positions in space as the pedicle screw is threaded into the vertebra. But the accuracy of the computer calculation is only as accurate as the trueness of the screw to instrument interface.

Another objective this invention is to improve the surgeon's security performing screw insertions with computerized navigation.

After the screw is safely threaded and secured within the pedicle to the surgeon's satisfaction and the screwdriver removed, the tulip can again be moved about the universal joint to accommodate a spinal stabilization rod. A series of screw/tulip assemblies and the rod are then secured together by retention nuts to complete the stabilization framework.

When people enter a strength exercise program to build muscle, it has been observed that bone growth and bone strength are also enhanced. The increased tendon and muscular strength exerts a stress that is beneficial to bone growth. Bone cells are programmed to recognize micro stresses and strains that form and renew bones Insufficient micro stresses and strains causes bone to resorbed or be replaced with soft tissue. Too much micro stresses and strains causes micro stress fractures that can also kill bone cells. Micro stresses and strains exerted upon bone, depending upon their magnitude, either create bone or renew healthy bone. This is referred to as the bone's mecanostate where just like a thermostat that turns on heat or air conditioning according to a specific temperature, bone generates new cells or renews old ones according to specific microstrain. Mono axial pedicle screws provide the surgeon with a good feel. However, they do not provide the many benefits of polyaxial assemblies. A principle objective of this invention is to provide the surgeon with a secure “feel” of monoaxial apparatus when using polyaxial assemblies. One of the principle objectives of this invention is to provide a spine stabilization structure that is as strong as prior art structures but has the ability to transmit a certain degree of stress to the spinal bones under treatment so as to enhance bone growth and strength.

In the present practice of spinal stabilization, many of the associated spinal rods are made of titanium and other extremely stiff materials. These materials are used because of their strength. However, when a stiff rod is subjected to stress, it will resist “give”. Little, if any, stress is imparted to the bones under treatment. This can cause a phenomenon called stress shielding where physiologic loads are propagated not through the bone, but instead around the bone and through the implant, causing insufficient micro stress and strain with associated bone resorption. In order to reduce the danger of stress shielding and propagate forces through the bone, applicant utilizes composite rods that are equal in strength to the stiffer metals but transform stress into elastic movement that gradually propagates stresses into the bone for bone formation and renewal. With a too stiff rod stress can be transferred to the weakest link in the chain; e.g., where the screw engages the bone. Pull-out can result. In order to reduce the danger of pull-out, and to stimulate surrounding bone with the proper bone cell producing micro stresses and strains, applicant utilizes composite rods that are equal in strength to the stiffer metals but can be designed to absorb a degree of stress because of their flexibility before that “stress” reaches the pedicle screw/bone interface. Thus, the dangers of pull-out are reduced.

Spinal stabilization operations are difficult and demanding on the operating physician. Because of the universal joint connection between the head of the screw and the interior of the tulip, looseness can develop between the driving instrument (the special polyaxial screwdriver) and the polyaxial screw. This is sometimes referred to as a “wobble effect” that can reduce the effectiveness of the operating physician. Wobble effect makes it difficult for the surgeon to thread a true and safe path into the unseen pedicle and it reduces the tactile “feed back” through the instrument that informs the surgeon if the screw is located in the softer inner or harder outer pedicle bone.

Another principle advantage of certain embodiments according to the present invention described herein is to establish, once the axis of the screw insertion is determined, a secure and true connection between the screw and the screwdriver that insures that the axis of the screwdriver is aligned and locked co-axially with the axis of the screw as rotation is imparted by the surgeon. This reduces or eliminates any wobble effect. This connection is most preferably accomplished by providing interengaging conical surfaces between the head of the screw and a driving shaft of the screwdriver.

Another principle advantage of such embodiments is that the interengaging conical surfaces engage the instrument tip and screw so that the tactile sensation of the screw tip to screwdriver handle is improved by propagating vibrations from the screw tip to instrument handle. This gives the surgeon a clearer situational awareness about the unseen screw by ensuring that the axis of the screw and instrument are truly and tightly aligned and the sensation caused by the soft inner bone tissue of the pedicle bone and the harder outer cortical bone tissue of the outer pedicle bone can be felt as different sensations through the instrument.

The drive shaft of a representative embodiment of the screwdriver is preferably equipped with a replaceable “screw engagement unit.” This allows the screw engagement unit to be made in harder steel than the rest of the instrument in order to be built to higher resistance and more precise specifications that are required to maintain a secure and true connection between the screw and the screwdriver. A replaceable “screw engagement unit” allows the rest of the instrument to be constructed in a less costly steel.

The replaceable “screw engagement unit” also improves the durability of the instruments good function. Instruments during the normal course of surgery can be accidently dropped upon a very hard operating or sterilization room floor and this can impact and bend the tip of the instrument that engages the screw. By making the “screw engagement unit” a separate part that is replaceable, impact on the tip will more likely only damage the replaceable unit and not harm the true axis of the entire instrument, which is an essential feature of its function. Should the tip wear, the screw engagement unit can be replaced while keeping the rest of the instrument.

One conventional screw driver for use with a spinal fixation system is, for example, disclosed in US 2006/0111712 A. This conventional screwdriver comprises a drive shaft attached to a handle and having a screw engaging end. The shaft is rotatable disposed within an elongated guide cylinder having at one end means to threadably engage the threads of the tulip of a tulip/screw assembly. As the elongated guide cylinder is on the outside, the surgeon has a tendency to grip it. If the surgeon rotates the handle to rotate the pedicle screw with one hand and grips the elongated guide element with the other hand, this can result in unscrewing the guide cylinder from the tulip and creating screw wobble making proper screw threading more and more difficult.

Another conventional screwdriver for spinal fixation systems is further disclosed in EP 1 946 711 A. The elongated guide element of such conventional screwdriver is provided with a locking button. This button is capable of automatically locking the shaft and therefore preventing it from unscrewing from the head of the polyaxial screw. When the screwdriver is used to unscrew, the button must be pressed to disengage the elongated guide element from the shaft to allow the elongated guide element to be rotated in reverse direction to remove the screw.

An exemplary embodiment of a screwdriver according to the present invention has a hollow grip telescopically disposed outside and in co-axial relationship with a drive shaft and an intermediate elongated tubular guide element. The drive shaft and the elongated guide element are independently rotatable with respect to one another about their co-located longitudinal axes and supported within the grip. The grip enables the surgeon to grasp the screwdriver and impart rotation to the pedicle screw without interfering with the connection between the guide element and the tulip. The screw engagement function is thus independently separated from the guide element-tulip connection and its attendant function. Thus, the surgeon can use the grip element to secure pedicle screw insertion without the danger of loosening the guide tulip engagement.

The replaceable screw engagement unit of certain embodiments of the present invention includes a tapered or conical screw engagement surface adapted to engage a matching tapered surface formed within a recess in the head of the pedicle screw. The engagement between these two tapered surfaces improves the ability to “true” the screw and the screwdriver in full co-axial engagement, while substantially eliminating the screw wobbles while improving the tactile sensation of the screw within the entire instrument.

The removable screw engagement elements are preferably provided with a suitably configured drive head, for example a hex or torx extension adapted to engage a correspondingly configured hexagonal or torx indentation within the head of the pedicle screws. Hex and torx embodiments of the drive head are preferred as each provides a positive drive between the drive shaft and the screw.

According to certain embodiments of the present invention, the grip has a rear end abutting a knob on the elongated guide element. The grip is longitudinally movable so it is capable of being shifted distally toward the engagement structure. During use, the grip can therefore be shifted distally until a distal end portion of the grip is disposed about and substantially covers the screw engagement unit. This helps shield the structures of the screwdriver tool at its distal end to thereby aid in the prevention of muscles or other tissue from becoming entangled and injured by the special screwdriver as the screw is threaded into bone. However, the grip can be manually shifted back from the shielded position in a proximal direction by the surgeon for inspection.

Another objective of certain embodiments of this invention is to provide a screwdriver that can be assembled and disassembled without a requirement for special tools. Especially designed structures of such embodiments also protect against accidental disassembly.

According to an embodiment of the present invention, the polyaxial bone screw assembly can include a fenestrated screw adapted to receive a bone cement injector. This embodiment is especially desirable when treating osteoporotic vertebra. The flow of bone cement through the screw is improved because the bone cement injector has a tapered outer surface adapted to engage the tapered recess surface formed in the head of the fenestrated bone screw. This improves the management of pressure through the cement injector and fenestrated screw system which is required to properly dose the amount of cement within an osteoporotic vertebra.

When treating patients with Osteoporosis and Osteopenia, it is advantageous to increase bone density. Even with bone density enhancements, the dangers of screw “pull-out” are ever present. A still further objective of this invention is to provide improved means to increase bone density by introducing bone density material through a fenestrated screw that preserves the advantages of the interengaging conical surfaces while combining that bone density advantage with a stabilizing rod that will absorb a limited amount of stress so that stress can be isolated from the screw/bone interface.

As mentioned previously, the present invention utilizes a rod made from a fiber-reinforced plastic. Such a composite rod can be used in combination with a solid screw or a fenestrated screw. The composite rod is preferably as strong but not as rigid, as a titanium rod having identical dimensions. Extreme stiffness is a disadvantage for pedicle fixation systems especially when used for treating osteoporotic vertebrae. Composite rods can be designed to be sufficiently strong to perform the required stabilizing function but with a degree of flexibility to isolate stress from the bone-screw interface and at the same time permitting a limited degree of stress to reach the bones under treatment. Additionally, a composite rod will not interfere with x-ray or other non-invasive inspections during the curative stage as do metallic rods.

### BRIEF DESCRIPTION OF THE DRAWINGS

Embodiments of the invention will be illustrated with reference to the following drawings, wherein like reference numerals through the various Figures denote like structural elements, and wherein:

FIG. 1 is a perspective view of a composite rod having a series of pedicle screw/tulip assemblies secured thereto,

FIG. 2A is a perspective view of a tulip employed in the pedicle screw/tulip assemblies shown in FIG. 1,

FIG. 2B is a cross-sectional view of the tulip along the line 2B-2B of FIG. 2A,

FIG. 3A is an exploded view of a pedicle screw/tulip assembly,

FIG. 3B is an assembled cross-sectional elevational view of the pedicle screw/tulip assembly depicted in FIG. 3A,

FIG. 4 is a side elevational view of the distal end of an exemplary screwdriver in accordance with an embodiment of the invention being assembled with a pedicle screw/tulip assembly,

FIG. 5A is a side elevational view of an exemplary screwdriver in accordance with an embodiment of the invention assembled with a screw/tulip assembly,

FIG. 5B is a perspective view in a distal direction of the exemplary screwdriver in accordance with an embodiment of the present invention but omitting the handle therefrom,

FIG. 6A is a side elevational view of an exemplary screwdriver similar to FIG. 5A but having the screw/tulip assembly that is assembled therewith turned through 90° and showing the grip thereof in an unshielded position,

FIG. 6B is a distal end view of the screwdriver depicted in FIG. 6A, but having the grip thereof shifted longitudinally into a distal shielding position,

FIG. 7A is a cross-sectional side view of the exemplary screwdriver and its assembled screw/tulip assembly as taken along line along the line 7A-7A of FIG. 6A,

FIG. 7B is an exploded cross-sectional side view of the exemplary screwdriver depicted in FIG. 7A,

FIG. 8A is a partial cross-sectional view of a proximal end of the exemplary screwdriver shown in a position whereby the guide and drive shafts are longitudinally fixed relative to one another,

FIG. 8B is a partial cross-sectional view of a proximal end of the exemplary screwdriver shown in FIG. 8 but in a position whereby the guide and drive shafts are released for longitudinal movement relative to one another,

FIG. 9 is an enlarged side elevational view similar to FIG. 4 but partly in cross-section, showing an exemplary screwdriver in accordance with an embodiment of the invention being assembled with a screw/tulip assembly.

FIG. 10A is a perspective view of the connection between the drive shaft and the screw engagement unit of the screwdriver,

FIG. 10B is a side view of showing the elements of the drive shaft and the screw engagement unit as depicted in FIG. 10A assembled to one another,

FIG. 11 is a cross-sectional view of a fenestrated screw and cement injector,

FIG. 12 is a cross-sectional view of the fenestrated screw in a bone,

FIG. 13 is a perspective view of another possible embodiment for the screw engaging unit,

FIG. 14 is a perspective of yet another possible embodiment of a screw engagement unit,

FIG. 15 is a side elevational view showing the embodiment of the screw engagement unit depicted in FIG. 14 connected operably to a pedicle screw/tulip assembly; and

FIG. 16 is an enlarged cross sectional view of the connected screw engagement unit and pedicle screw/tulip assembly as depicted in FIG. 15.

### DETAILED DESCRIPTION

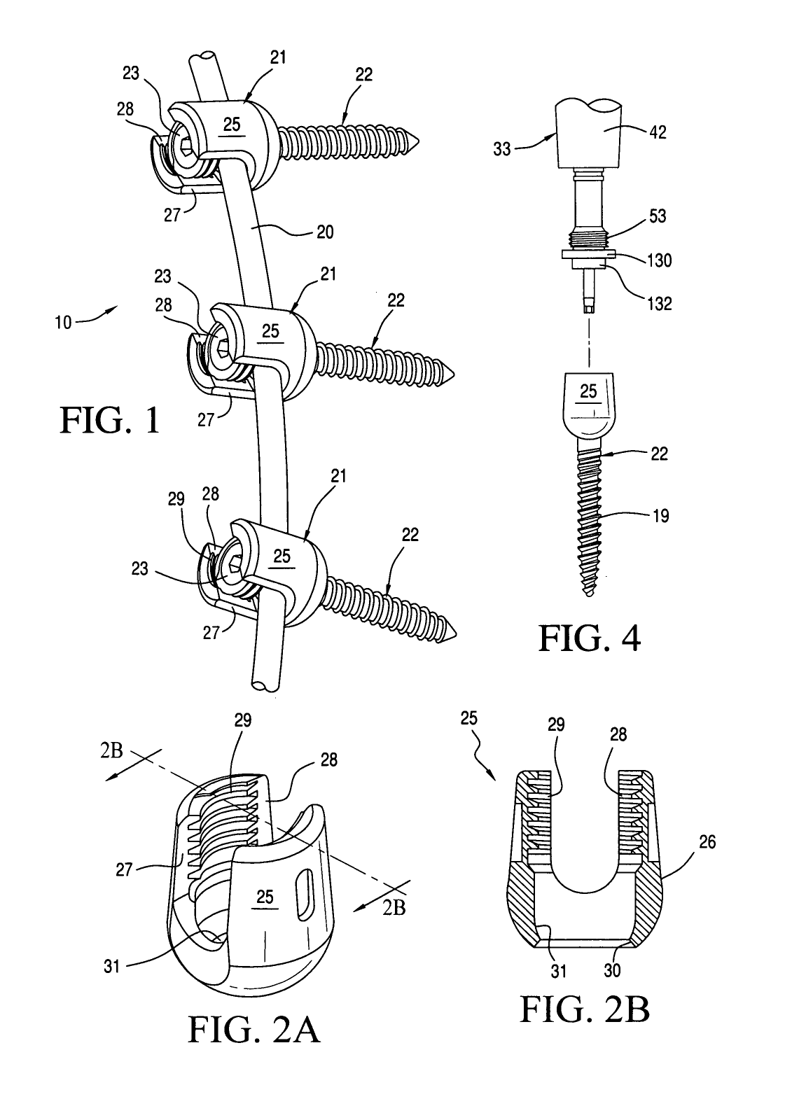

An exemplary spinal fixation system 10 is shown in accompanying FIG. 1. As depicted, the system 10 includes a composite rod 20 that is as strong as metallic spinal rods of like cross-sectional dimensions and length but, depending on the length, characteristics and amount of fiber embedded in the plastic, can be engineered to permit limited degrees of flexibility for reasons hereinafter described in more detail. See in this regard, U.S. Patent Application Publication Nos. 2008.0262548 and 2010/0042163 (the entire contents of each being expressly incorporated hereinto by reference).

As is well known in the art, the rod 20 will have a contour generally the same as that portion of the spine to be treated and will ultimately be located in a position generally parallel to that portion of the spinal column under treatment.

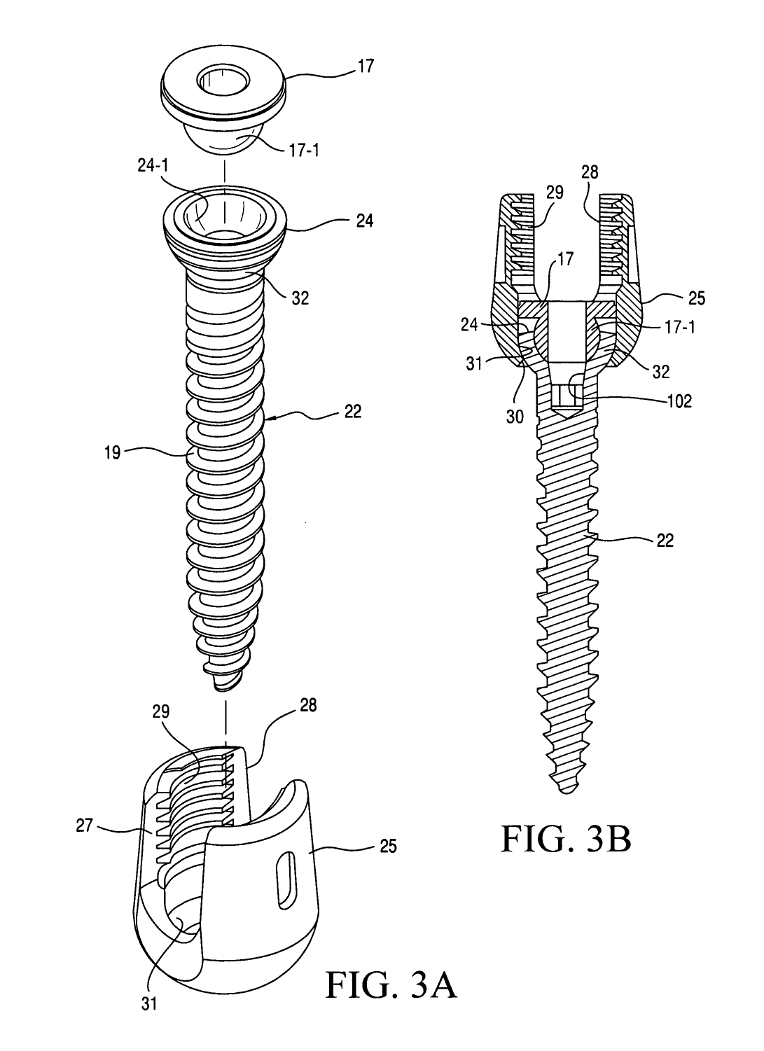

A series of pedicle screw/tulip assemblies 21 are secured to the rod 20. The screws 22 thereof are polyaxial—that is, their heads 24 are mounted for universal movement within tulips 25. In this regard, a crown 17 is press fitted into each tulip (see FIGS. 3A and 3B). Operationally, the screws 22 are threadably affixed to an appropriate vertebrae bone by a screwdriver tool in accordance with the present invention which will be described in greater detail below and which is generally indicated by reference numeral 33 (see FIG. 4). Subsequently, the rod 20 is placed within the grooves 27, 28 and the stabilization is completed by threadably securing nuts 23 within the threaded upper interior wall 29 of tulip 25 so it bears against an upper region of the rod 20.

As is perhaps more clearly seen in FIGS. 2A and 2B, the tulip 25 has a cylindrical wall 26 that is grooved at 27 and 28 to receive the rod 20. The wall 26 is interiorly threaded at 29. The upper head 24 of the screw has a recessed bowl shaped region 24-1 that receives conformably shaped protruding bowl shaped section 17-1 of crown 17. The crown 17 thus engages the recessed bowl shaped region 24-1 of the head 24 of the pedicle screw 22.

The threaded portion 19 of the pedicle screw 22 extends outwardly beyond the tulip 25 through the lower opening 30 thereof. The head 24 of the screw 22 also defines an exterior convexly curved region 32 which mateably cooperates with a mating interior bowl shaped region 31 at a lower end of the tulip 25 which surrounds the opening 30. The cooperatively mated bowl shaped surfaces 31 and 32 thereby interface to provide a limited universal ball-type joint connection between the pedicle screw 22 and the tulip 25.

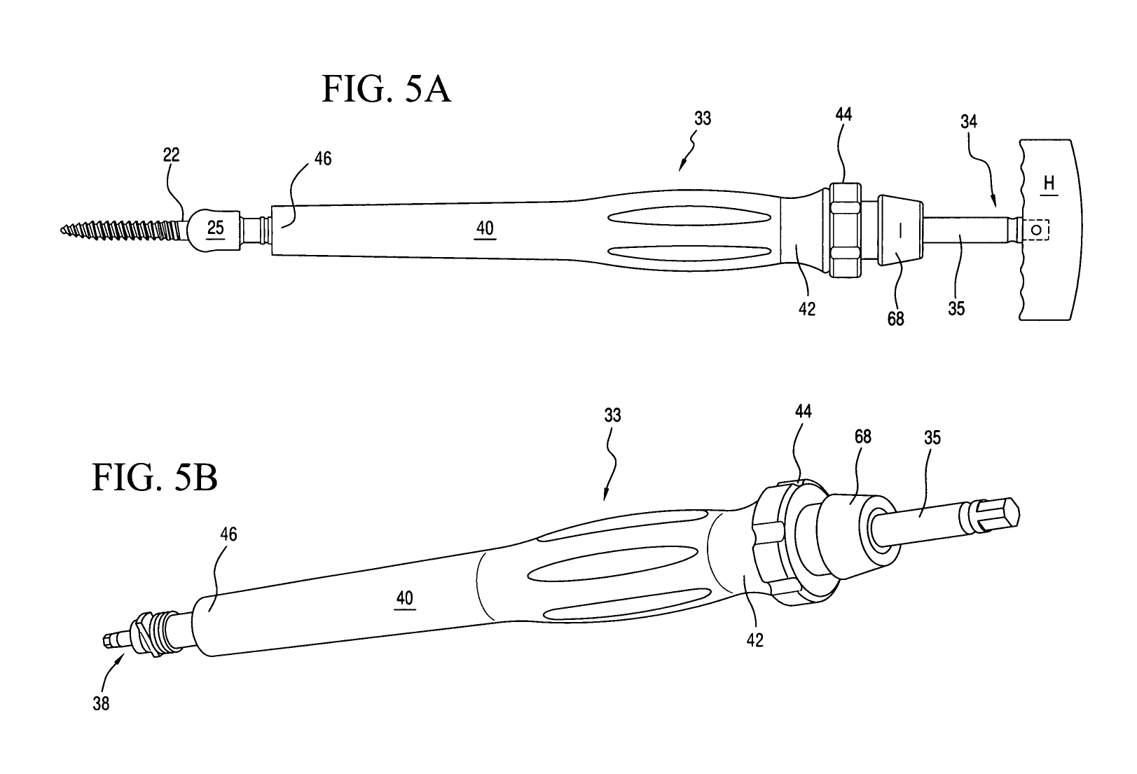

As noted briefly above, the implantation of the pedicle screw 22 is accomplished through the use of a screwdriver tool 33 in accordance with an aspect of the present invention. As shown particularly in FIG. 5A through FIG. 7B, the screwdriver tool 33 includes a longitudinal drive shaft 35 having a proximal end 34 to which handle H is secured, and a distal end 37 to which a screw engagement unit 38 (see FIGS. 10A and 10B) is releasably coupled.

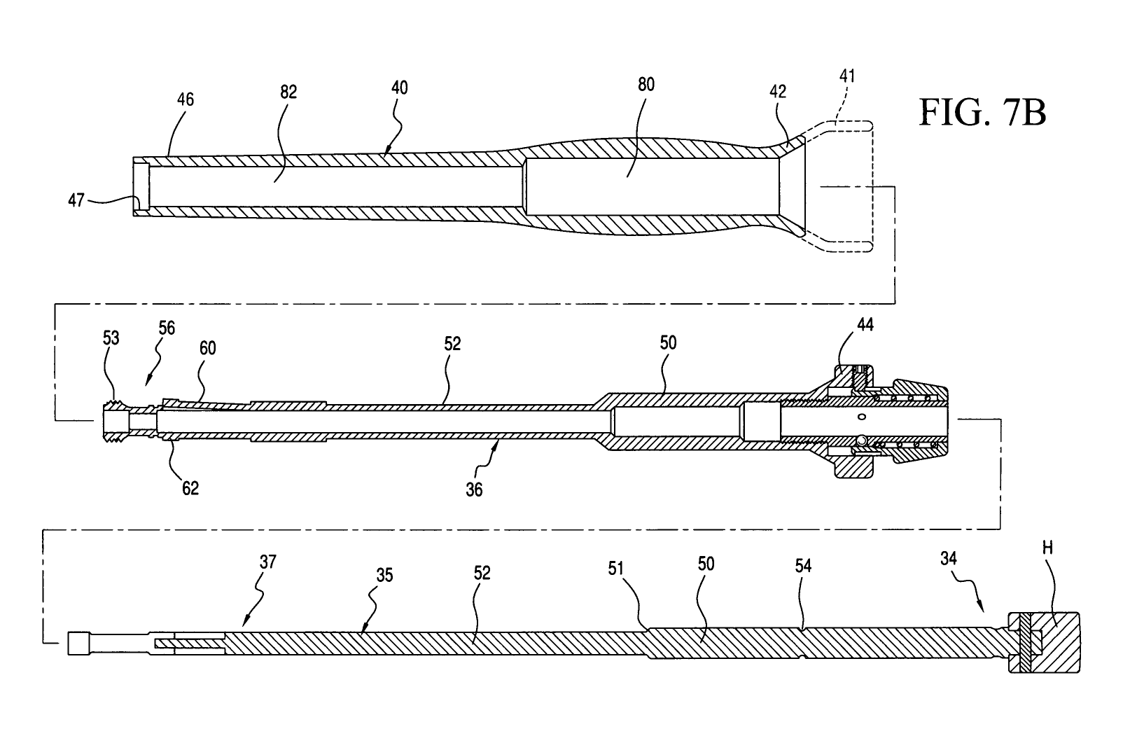

The drive shaft 35 is coaxially received within a tubular guide member 36 so that each is capable of independent longitudinal and rotational movements relative to the other. In addition, the drive shaft 35 and guide member 36 are coaxially housed within an outer grip 40. Thus, the grip 40 is most preferably in the form of a hollow generally cylindrically shaped structure which is sleeved in coaxial relationship around the drive shaft 35 and the cylindrical guide member 36. The grip 40 has a proximal end 42 which, in a first position, abuts knob 44 attached to a proximal end of the elongated guide member 36. The grip 40 also has a distal end 46 which is counterbored at 47 (see FIG. 7B).

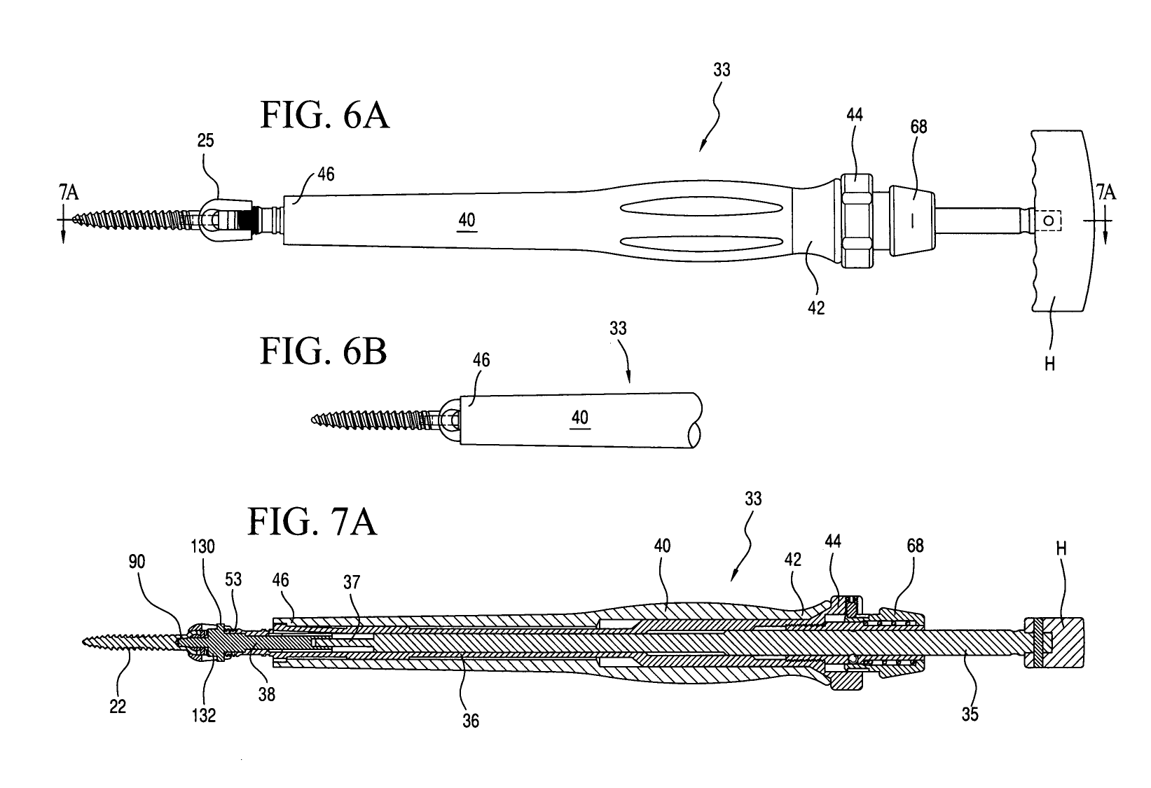

In operation, the grip 40 can be shifted longitudinally in a distal direction to cover the screw engaging unit 38 (see FIG. 6b) or moved oppositely in a proximal direction for inspection by the operating physician (see FIG. 6A). In such a manner, therefore, the distal end 46 of the grip 40 will cover the distal operative structures associated with the screw engagement unit 38 to thereby shield surrounding tissue therefrom. Although not shown in the Figures, while in such a distally shifted position, the proximal end 42 will be spaced longitudinally from the knob 44. According to some embodiments, however, the grip 40 may have an expanded extension 41 that covers knob 44 (shown by the dotted lines in FIG. 7B) so as to ensure that knob 44 will not be used when handle H should be used.

FIG. 7B shows an exploded view of the three principal longitudinal members of the screwdriver 33, namely; the drive shaft 35, the guide member 36 and the outer grip 40. The drive shaft 35 has a first proximal section 50 and a second distal section of lesser diameter 52 joined to one another by a conical area 51. Along the length of section 50 there is the slight circumferential groove 54.

The guide member 36 is provided at its distalmost end 56 with an exterior threaded portion 53. The threaded portion 53 is adapted to threadably engage the interior threads 29 of the tulip 25 as it approaches crown 17 in response to turning movements being applied to the knob 44. At a short distance proximally spaced from threads 53, the guide member 36 is formed with a flexible retainer leaf spring 60 that is biased outwardly. A collar 62 is formed about that portion of the guide member and spring. The collar 62 is adapted to be received in the counterbore 47 formed at the distal end 46 of the grip 40.

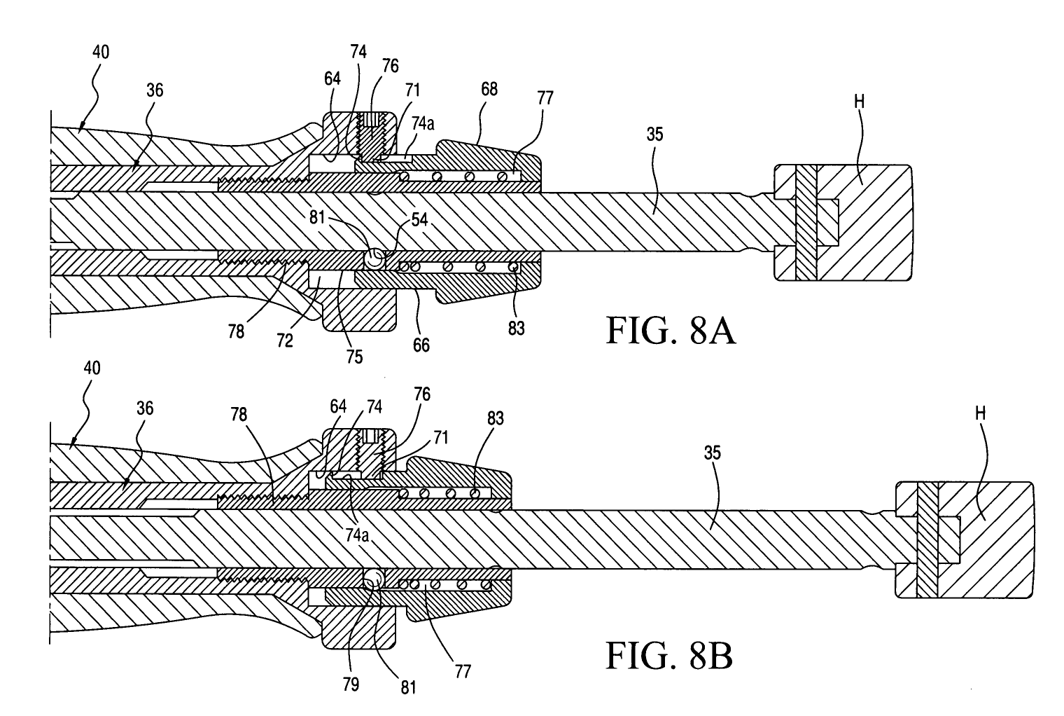

As has been described briefly above, the proximal end of the guide member 36 includes an enlarged circumferential knob 44. As shown in FIGS. 8A and 8B, a central coaxially located counterbore 64 is formed through knob 44. A skirt 66 of a cap 68 is slidably received in the counterbore 64. Disposed radially in knob 44 is a threaded set screw 76 that has a portion 71 extending into the counterbore 64 and received by a slot 74a of the skirt 66. The inner end of skirt 66 is formed with a ridge or lug 74 that engages portion 71 when the cap is in its locked position. The cap 68 also forms an annular chamber 72 with the exterior 75 of a cylindrical sleeve 78. The sleeve 78 is threadably secured at its distal end to the guide member 36. The sleeve 78 has an aperture 79 that carries a ball 81. In the locked position, ball 81 is partially received in the circumferential groove 54 of drive shaft 35 which in turn prevents relative longitudinal movement to occur between drive shaft 35 and guide cylinder 36. Such a locked position is shown in FIG. 8A. The cap 68 and sleeve 78 also form an annular chamber 77 that receives a spring 83 which exerts a bias force against the cap 68 toward handle H so as to retain the cap 68 in its locked position. Movement is therefore restrained by the engagement of lug 74 against the set screw 76. As long as ball 81 is retained within the groove 54, the elements 35 and 36 are longitudinally fixed relative to one another.

When the cap 68 is longitudinally moved in a distal direction (i.e., moved to the left as seen in FIG. 8B) against the bias force of the spring 83 into an unlocked position, a portion of the chamber 77 will then responsively be positioned adjacent the ball 81. As such, the ball 81 is no longer restrained by groove 54 but instead may be disengaged from the groove 54 and received partially within the chamber 77. Once the engagement between the groove 57 and ball 81 ceases, the drive shaft 35 can be then be removed physically from the screwdriver 33 by manually pulling the shaft in a proximal direction. After the drive shaft 35 is removed, the guide cylinder 35 can then likewise be removed in the same direction. Since cap 68 is moved inwardly before disassembly can occur, it is therefore virtually impossible for disassembly to occur accidentally.

The interior of grip 40 has an enlarged chamber 80 to receive the enlarged section 50 of the guide member 36. The interior of grip 40 also has a reduced chamber 82 to receive the reduced section 52 of the guide member 36.

The assembled elements of FIG. 7A depict the manner in which the guide cylinder 36 is threadably received in the tulip 25. FIG. 7A also depicts a hexagonal extension 90 of drive shaft 35 being received operably in a hexagonal recess 92 of the pedicle screw. In operation, the surgeon may attach the threaded portion 53 of the guide member 36 by applying manual turning movement thereto via knob 44 independently of the drive shaft 36. Once the threaded portion 53 is securely threadably mated to the tulip 53, the surgeon may then seat the hexagonal (or other) extension 90 of the drive shaft within the hexagonal (or other) recess 92 so that turning movements applied via the handle H can then be transferred to the screw 22 to cause the threads 19 to become embedded within a pedicle of a vertebra bone.

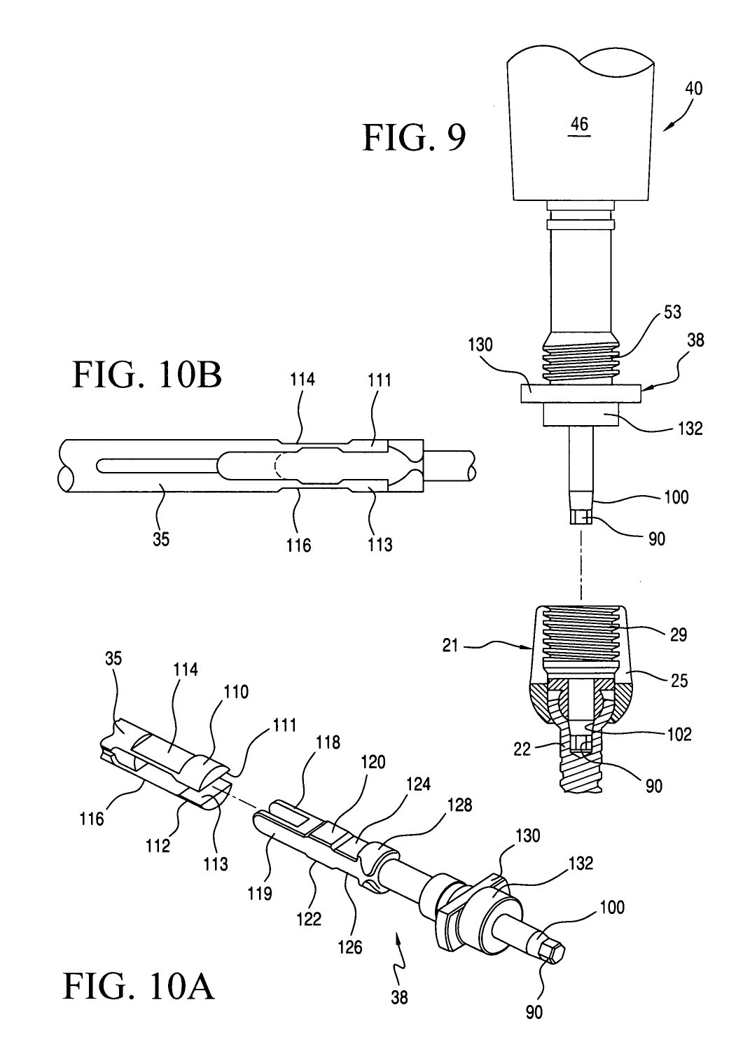

FIGS. 10A and 10B depict in greater detail one embodiment of the screw engagement unit that may be coupled removably to a distal end of the drive shaft 35. In this regard, FIG. 10A is an exploded side elevational view showing the screw engagement unit 38 prior to insertion in a tulip assembly 21. Of particular note is the conical surface 100 of the screw engagement unit 38 which conformably matches the conical surface 102 in the head 24 of the pedicle screw (see FIG. 9). Also of note is the hexagonal extension 90 of the screw engagement unit which operably mates with the conformably shaped the hexagonal recess 92 in the pedicle screw. When the extension 90 is inserted into recess 92, the surfaces 100 and 102 are thus fully engaged. This engagement of the mated surfaces 100, 102 thereby “trues” the screw 22 with the screwdriver 33 to eliminate or greatly reduce wobble.

The connection between the drive shaft 35 and the screw engagement unit 38 can also be seen in FIGS. 10A and 10B. At the end of shaft 35 are two fingers 110 and 112. Finger 110 is formed with an exterior depression 114 and finger 112 is formed with an exterior depression 116. The fingers are formed with opposing interior projections 111 and 113.

The screw engagement unit 38 is most preferably formed with a pair of fingers 118 and 119 which are adapted to fit into the space between fingers 116 and 118. Disposed a short distance from fingers 118 and 119 are upper and lower ridges 120 and 122 that form upper and lower depressions 124 and 126 with collar 128.

When engaged, the projections 111 and 113 are received respectively in depression 122 and 124 to form a readily removable connection between the drive shaft 35 and the screw engagement unit 38 because of finger flexibility. However, rotation is secure because of the interlocking fingers. This arrangement provides a quick means to replace units 35 with units of various steel hardness.

Between the connection area and the conical surface 100, the screw engagement unit 38 is formed with a generally rectangular member 130 for reception in the tulip grooves 27 and 28. A circular member 132 is formed next to the member 130. Member 132 engages the interior surfaces of the tulip 25 when the unit 38 and the pedicle screw are engaged.

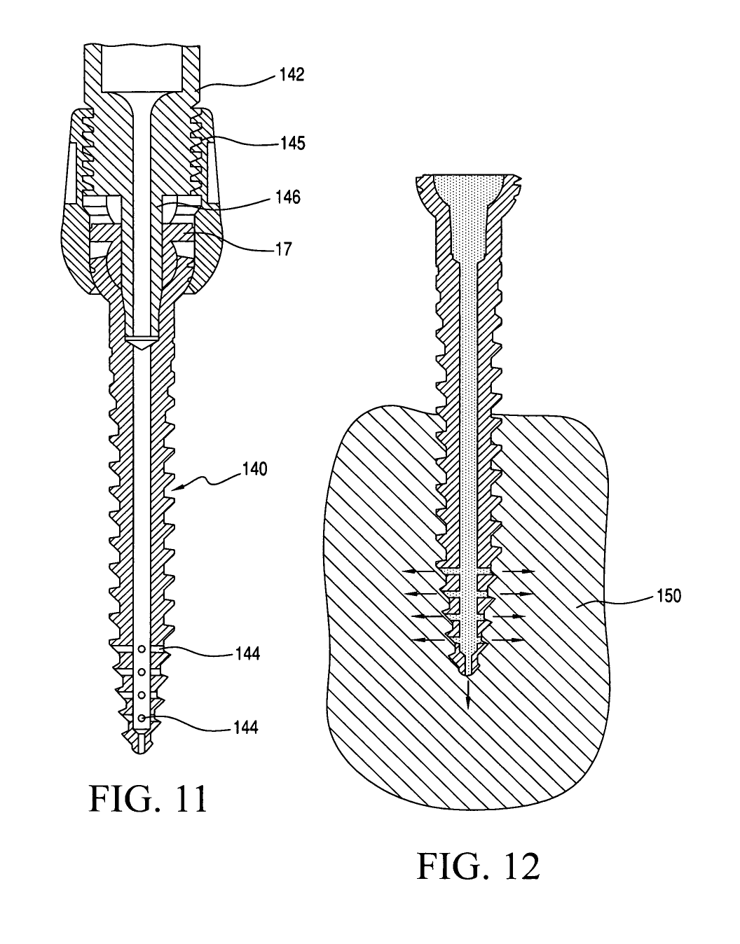

As mentioned earlier, it is oftentimes useful and/or necessary to strengthen osteoporotic vertebrae prior to constructing the stabilizing structure. FIGS. 11 and 12 depict an embodiment of a fenestrated pedicle screw 140 that is adapted to receive a bone cement injector 142. As seen in FIG. 11, the pedicle screw 140 is hollow throughout most of its length and has a plurality of radial openings 144 in fluid communication with the hollow.

The injector 142 is externally threaded at 145 to be received by the interior threads of the tulip. A conduit 146 is received in the recess normally receiving the screw engagement unit. The lower end of the injector is formed with an exterior that is adapted to be received by a torx or hexagonal drive. As is well known in the art, cement may be forced into the fenestrated screw and into the bone 150 through the radial openings 152.

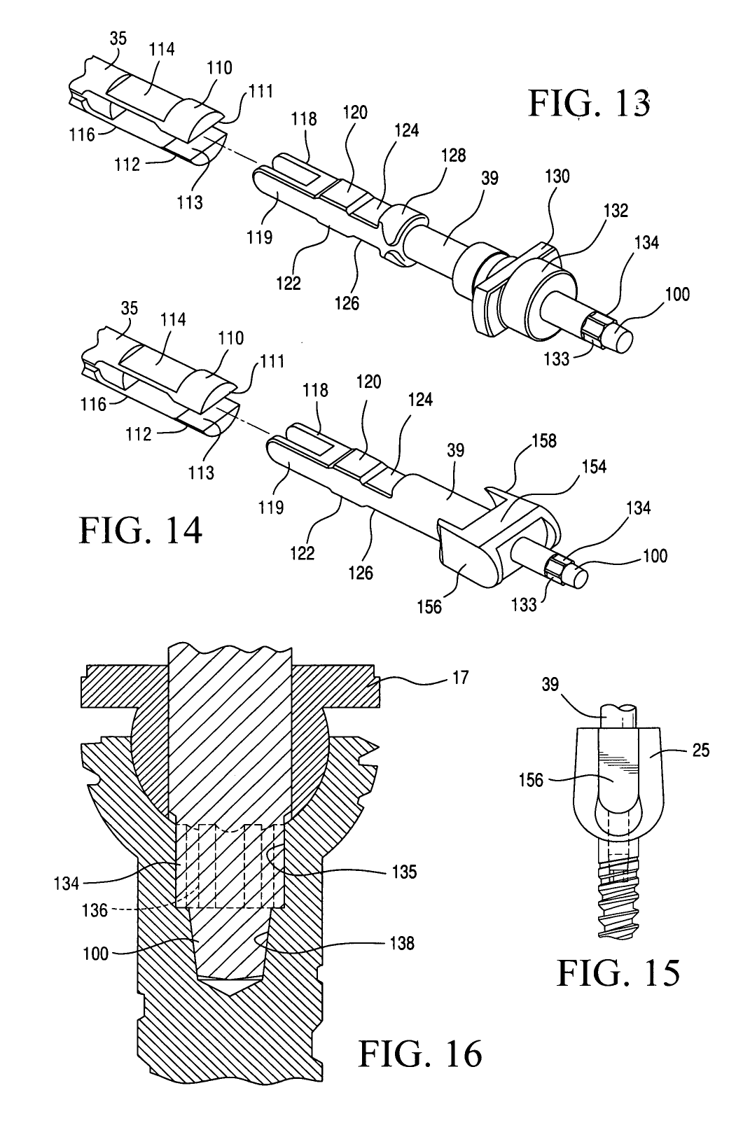

FIGS. 13 and 14 depict other embodiments of the screw engagement unit 38 that may be employed. In the embodiments depicted, the inter-locking finger arrangement of FIG. 1 remains the same. However, in the embodiment of FIG. 14, the tulip engagement structures have been modified. Specifically, a body 154 is secured to shaft portion 39. On either side of the body are wings 156 and 158 that end in curved edges that are the same as the bottom curves of grooves 27 and 28 of the tulip 25 and such that the side edges of the wings 156, 158 engage the sides of these grooves 27, 28, respectively. (See FIG. 15)

As shown in FIGS. 13 and 14, the hexagonal positive drive extension associated with the screw engagement unit 28 described previously has replaced by a torx 134 having a plurality of ribs 133. The pedicle screw head 24 is formed with a recess 135 having grooves 136 to receive each of the ribs. (See FIG. 16) In this embodiment the conical surface 100 is distal of the torx and a corresponding conical recess 138 is formed in the head of the pedicle screw below the recess 135. Many surgeons prefer this arrangement although both embodiments provide a firm connection between the screwdriver and the pedicle screw.

There has thus been described above a screwdriver 33 that has an outer grip 40, a cylindrical guide member 36 that has means to independently threadably engage and disengage with the tulip 25 and a drive shaft 35 that can independently impart rotation to the pedicle screw 22 without interfering with the guide member 36 and tulip 25 connection. While preserving these functions it is also important for the operating physician to be able to quickly disassemble and assemble the screwdriver for cleaning, inspection and replacement of parts.

In use, the attending surgeon will preoperatively prepare the unit as depicted in FIG. 5A. That is, the guide member 36 will initially be threadably connected to the tulip 25 by rotating it via knob 44 so as to cause the threaded portion 53 of the guide member 36 to be threadably coupled to the threads 29 of the tulip 25. The screw engagement unit 38 fixed to drive shaft 35 will then be accurately located with respect to the pedicle screw 22 via the inter-engaging conical surfaces 100 and 102 and is in a driving relationship via either the hexagonal or torx arrangements described above.

The surgeon will place the threaded shank 19 of the pedicle screw 22 against a pre-selected vertebra while holding grip 40 with one hand and imparting a rotary motion via handle H with the other. The guide member 36 that is connected to the tulip 25 will not be affected by such turning movement applied to the drive shaft 35 since the latter is capable of independent rotational motion relative to the former. Prior, to rotation, the surgeon may optionally move grip 40 in a distal direction away from knob 44 so it assumes a shield position and thereby protect surrounding tissue from most of the rotating parts.

After the first pedicle screw is secure, the guide member 36 is counter-rotated via the knob 44 so as to disengage the threaded portion 53 from the threads 29 of the tulip 25. When disengaged, the pedicle screw/tulip assembly 21 remains in place and the screwdriver may withdrawn. The physician will then quickly engage the drive shaft with a second screw/tulip assembly 21 and secure it into a second selected vertebra. This process is repeated until all required pedicle screws 22 are secured to selected pedicles. In all operations, time is of the essence. A quick replacement of multiple screw/tulip assemblies is thus essential.

The framework for the system 10 is then completed by threading retention nuts 23 against the rod.

Operationally, the same steps are taken whether one is using the torx or hexagonal engagement means.

In some instances, spinal reconstruction must be performed on patients with osteoporotic vertebrae. The advantages already described can be preserved by utilizing a fenestrated screw/tulip assembly engineered for reception by bone cement injector 142 as described above with respect to FIGS. 11 and 12. The fenestrated screw retains the universal movement with its tulip and the injector is threaded so as to engage with the interior threads of the tulip.

The embodiments described herein introduce to the physician a combination of a screwdriver and a pedicle screw/tulip assembly that when employed collectively with the composite rod increases the ability to secure accurately a pedicle screw by eliminating wobble, improve the tactile sensations of the screw through the screwdriver, eliminate the problem of accidentally unscrewing the guide cylinder by providing an outer grip, provide the grip with a position to cover many of the rotating ports so as to protect surrounding muscle and tissue, a rod that enhances the ability to check progress of tissue formation or resorption without invasive surgery and is easily assembled and disassembled so as to replace parts when necessary.

In addition to the above, the embodiments described herein provide improved means to introduce bone cements into a porous bone prior to constructing the stabilization framework.

The preferred embodiments have been described and illustrated, but the specific forms and arrangement of parts should not be limiting, and the following claims define what is to be secured and protected.

1. A screwdriver for securing a pedicle screw of a screw/tulip spinal fixation assembly into a vertebra comprising:
a tubular guide member having a distal threaded portion for threadable coupling with a tulip associated with the screw/tulip assembly;
a drive shaft coaxially received within guide member for independent longitudinal and rotational movements therewith; and
a screw engagement unit attached to a distal end of the drive shaft for operative engagement with the pedicle screw.

2. The screwdriver of claim 1, further comprising an outer generally cylindrical grip sleeved around the guide member.

3. The screwdriver of claim 2, wherein the grip is longitudinally moveable between proximal and distal positions so as to uncover and cover, respectively, at least a portion of the screw engagement unit at the distal end of the drive shaft.

4. The screwdriver of claim 1, wherein the screw engagement unit includes a conical surface adapted to engage a conformably shaped conical surface of the pedicle screw.

5. The screwdriver of claim 1, wherein the screw engagement unit includes a drive engagement surface engaging a conformably shaped drive engagement recess of said pedicle screw.

6. The screwdriver of claim 5, wherein the pedicle screw includes a head formed with a hexagonally shaped recess and wherein the drive engagement surface has an exterior hexagonal surface conformably engageable with the hexagonally shaped recess of the pedicle screw head.

7. The screwdriver of claim 5, wherein the pedicle screw includes a head formed with a recess having longitudinal grooves along its length and wherein the drive engagement surface comprises a torx having ribs received by the longitudinal grooves of the recess.

8. The screwdriver according to claim 1, comprising a locking assembly which prevents independent longitudinal movements between the guide member and the drive shaft.

9. The screwdriver according to claim 1, wherein the locking assembly comprises:
a circumferential groove defined in the drive shaft,
a cap defining a chamber and coupled to the guide member, and
a ball carried by the cap and engageable with the groove when the cap is in a locked position to prevent independent longitudinal movements between the guide member and the drive shaft, wherein
the cap is moveable into an unlocked position wherein the chamber is positioned adjacent the ball so that the ball is received therein and disengaged from the groove whereby independent longitudinal movements between the guide member and drive shaft are permitted.

10. The screwdriver according to claim 9, wherein the guide member includes a counterbore, and wherein the cap includes a skirt slidably received within the counterbore of the guide member to permit movements of the cap between the locked and unlocked positions thereof.

11. The screwdriver according to claim 10, wherein the skirt includes a lug at an end thereof, and a slot proximally of the lug, and wherein the locking assembly includes a set screw having a portion thereof extending into the slot.

12. The screwdriver according to claim 1, wherein the locking assembly includes a spring for exerting a bias force to move the cap into its locked position.

13. The screwdriver according to claim 12, wherein the cap is moveably against the bias force of the spring into the unlocked position thereof.

14. A spinal fixation kit, which comprises:
a plurality of polyaxial screw/tulip assemblies which include a polyaxial screw receivable within a threaded tulip having an interiorly threaded portion, and
a connecting rod for interconnecting the plurality of screw/tulip assemblies, and
a screwdriver as in claim 1 for operative engagement with the screw/tulip assemblies.

15. A unit assembly comprising a pedicle screw/tulip assembly having a pedicle screw received within an interiorly threaded tulip, and a screwdriver for implanting the pedicle screw into a vertebrae, wherein the screw driver comprises:
an elongated tubular guide member having proximal and distal ends, and including a knob at the proximal end thereof to allow for rotational motion to be imparted thereto, and a threaded tulip engaging portion at the distal end thereof for threaded engagement with the interior threads of the tulip member;
an elongated drive shaft received within the guide member for independent longitudinal and rotational movements therewith, wherein the drive shaft includes a proximal handle end to allow rotation to be imparted to the drive shaft, and a distal pedicle screw engaging end opposite to the handle end; and
a grip sleeved around the guide member and drive shaft and being longitudinally movable between a distal position wherein a distal end of the grip covers at least a portion of the screw engaging end of the drive shaft, and a proximal position wherein the portion of the screw engaging end is uncovered.

16. The unit assembly of claim 15, wherein the screw engaging end of the drive shaft is formed with an exterior conical surface, and wherein the pedicle screw is formed with a bore formed with an interior conical surface that conformably mates with the exterior conical surface of the drive shaft.

17. The unit assembly of claim 15, wherein said screw engaging end of the shaft is formed with an exterior multi-face drive extension, and wherein the pedicle screw is formed with a recess having a multi-face surface that conformably receives the exterior multi-face drive extension.

18. The unit assembly of claim 17, wherein the exterior multi-face drive extension is hexagonal

19. The unit assembly of claim 17, wherein the exterior multi-face drive extension is a torx.

20. A spinal fixation system for stabilizing a spinal segment comprising:
a plurality of polyaxial screw/tulip assemblies which include a polyaxial screw receivable within a threaded tulip having an interiorly threaded portion,
a connecting rod for interconnecting the plurality of screw/tulip assemblies, and
a screwdriver for implanting the polyaxial screws of the screw/tulip assemblies in a respective vertebrae of the spinal segment in need of stabilization, wherein the screwdriver comprises:
(i) a tubular guide member having a distal threaded portion at a distal end thereof for threadable coupling with the interiorly threaded portion of the tulip of the screw/tulip assembly;
(ii) a drive shaft coaxially received within guide member for independent longitudinal and rotational movements therewith;
(iii) a screw engagement unit attached to a distal end of the drive shaft for operative engagement with the pedicle screw; and
(iv) an outer generally cylindrical grip sleeved around the guide member.

21. A spinal fixation system according to claim 20, wherein the screw engaging unit is formed with a first screw engagement tapered surface, and wherein the screw includes a head having a recessed second tapered surface conformably matching the first screw engagement tapered surface.

22. A spinal fixation system according to claim 21, wherein the screw engagement unit has a hexagonal segment adapted to be received by a corresponding hexagonal recess within the head of the screw

23. A spinal fixation system according to claim 20, wherein said screw engagement unit is releasably connected to the distal end of the drive shaft.

24. A spinal fixation system according to claim 20, wherein the connecting rod is formed of a composite material sufficiently resilient to transmit a stress to the stabilized spinal segment but sufficiently strong to retain spinal stability.

25. A spinal fixation system according to claim 20, wherein the grip includes a counterbore at a distal end thereof, and wherein the distal end of the guide member includes a resilient tongue, and a collar disposed about the tongue to engage the counterbore.
